SEQUENCE LISTING (4 sequences)

SEQ ID NO: 1 - DNA, 1287 bp, artificial sequence
  cDNA sequence _ seq signal B2m/Beta2m/Linker/Galpha1/Galpha2/Galpha3/intron4
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgaccttgtcacagcccaagatagttaagtgggatcgagacatgggaggtggcggatccggaggtggcggatccggaggtggcggatccggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggaggaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctc

SEQ ID NO: 2 - protein, 433 aa, artificial sequence
  Amino acid sequence of HLA-G5/Beta 2m.
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEEEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKAVASKEGDGGIMSVRESRSLSEDL

SEQ ID NO: 3 - DNA, 22 bp, artificial sequence
  primers_“ B2Msig TOPO sens ”
caccatgtctcgctccgtggcc

SEQ ID NO: 4 - DNA, 90 bp, artificial sequence
  primers _ “alpha3-i4-Xho-Stop anti sens ”
atcttaactcgagaggtcttcagagaggctcctgctttccctaacagacatgatgcctccatctccctccttactccatctcagcatgag

(19) United States
(12) Patent Application Publication
(10) Pub. No.: US 2011/0274711 A1
(43) Pub. Date: Nov. 10, 2011

(54) HLA-G POLYPEPTIDES AND PHARMACEUTICAL USES THEREOF

(75) Inventors: Benoit Favier, Paris (FR); Edgardo D. Carosella, Paris (FR); Joël Lemaoult, Melun (FR)

(73) Assignees: COMMISSARIAT A L'ENERGIE ATOMIQUE ET AUX ENERGIES ALTERNATIVES, PARIS (FR); HLA-G TECHNOLOGIES, LYON (FR)

(21) Appl. No.: 13/127,799

(22) PCT Filed: Nov. 4, 2009
(86) PCT No.: PCT/EP2009/064577

§ 371 Date: Jul. 28, 2011

Related U.S. Application Data
(60) Provisional application No. 61/112,816, filed on Nov. 10, 2008.

(30) Foreign Application Priority Data
Nov. 7, 2008 (EP) 08168677.6

(51) Int. Cl. A61K 38/17; C07H 21/00; C12N 15/63; C12N 5/10; A61P 29/00; C12N 1/15; C12N 1/19; C12P 21/00; A61P 37/06; C07K 19/00; C12N 1/21

(52) U.S. Cl. 424/192.1; 530/394; 536/23.4; 435/320.1; 435/325; 435/348; 435/419; 435/252.3; 435/252.33; 435/254.11; 435/254.2; 435/69.3

(57) ABSTRACT

The present invention relates to novel proteins and pharmaceutical uses thereof The invention more specifically relates to novel proteins comprising the sequence of an HLA-5 antigen fused to the sequence of a b2 microglobulin. The invention also relates to methods of producing such polypeptides, pharmaceutical compositions comprising the same, as well as their uses for treating various diseases including organ/tissue rejection.

The present invention relates to a novel protein and pharmaceutical uses thereof. The invention more specifically relates to a novel fusion protein comprising a domain of an HLA-G5 antigen fused to a B2 microglobulin. The invention also relates to methods of producing such a protein, pharmaceutical compositions comprising the same, as well as their uses for treating various diseases including organ/tissue rejection.

### BACKGROUND

Major histocompatibility complex (MHC) antigens are divided up into three main classes, namely class I antigens, class II antigens (HLA-DP, HLA-DQ and HLA-DR), and class III antigens.

Class I antigens comprise conventional antigens, HLA-A, HLA-B and HLA-C, which exhibit 3 globular domains ([alpha]1, [alpha]2 and [alpha]3), as well as unconventional antigens HLA-E, HLA-F, and HLA-G.

HLA-G is a non-classic HLA Class I molecule expressed by extravillous trophoblasts of normal human placenta and thymic epithelial cells. HLA-G antigens are essentially expressed by the cytotrophoblastic cells of the placenta and function as immunomodulatory agents protecting the foetus from the maternal immune system (absence of rejection by the mother). The sequence of the HLA-G gene has been described (e.g., Geraghty et al. Proc. Natl. Acad. Sci. USA, 1987, 84, 9145-9149 ; Ellis; et al., J. Immunol., 1990, 144, 731-735) and comprises 4396 base pairs. This gene is composed of 8 exons, 7 introns and a 3′ untranslated end, corresponding respectively to the following domains: exon 1: signal sequence, exon 2: alphal extracellular domain, exon 3: alpha2, extracellular domain, exon 4: alpha3 extracellular domain, exon 5: transmembrane region, exon 6: cytoplasmic domain I, exon 7: cytoplasmic domain II (untranslated), exon 8: cytoplasmic domain III (untranslated) and 3′ untranslated region.

Seven isoforms of HLA-G have been identified, among which 4 are membrane bound (HLA-G1, HLA-G2, HLA-G3 and HLA-G4) and 3 are soluble (HLA-G5, HLA-G6 and HLA-G7) (see e.g., Carosella et al., Blood 2008, vol. 111, p 4862).

The mature G1 protein isoform comprises the three external domains (α1-α3), the transmembrane region and the cytoplasmic domain.

The G2 protein isoform does not comprise the α2 domain, i.e., the α1 and α3 domains are directly linked, followed by the transmembrane domain and the cytoplasmic domain.
The G3 protein isoform lacks both the α3 and α3 domains, i.e., it comprises the α1 domain directly linked to the transmembrane domain and the cytoplasmic domain.
The G4 protein isoform lacks the α3 domain, i.e., it comprises the α1 domain, the α2 domain, the transmembrane domain and the cytoplasmic domain.

Soluble HLA-G isoforms all lack the transmembrane and cytoplasmic domains. More specifically:

The G5 protein isoform contains the α1, α2 and α3 domains, as well as an extra C-terminal peptide sequence of 21 amino acid residues encoded by intron 4 (as a result of intron 4 retention after transcript splicing and RNA maturation).
The G6 protein isoform corresponds to the G5 without α2, i.e., HLA-G6 contains α1 and α3 domains, as well as an extra C-terminal peptide sequence of 21 amino acid residues encoded by intron 4 (as a result of intron 4 retention after transcript splicing and RNA maturation).
The G7 protein isoform contains only the alphal domain, as well as 2 additional C-terminal amino acid residues encoded by intron2 (as a result of intron 2 retention after transcript splicing and RNA maturation).

All of these isoforms have been described e.g., in Kirszenbaum M. et al., Proc. Natl. Acad. Sci. USA, 1994, 91, 4209-4213; European Application EP 0 677 582; Kirszenbaum M. et al., Human Immunol., 1995, 43, 237-241; Moreau P. et al., Human

Immunol., 1995, 43, 231-236).

Previous studies have shown that HLA-G proteins are able to inhibit allogeneic responses such as proliferative T lymphocyte cell response, cytotoxic T lymphocytes mediated cytolysis, and NK cells mediated cytolysis (Rouas-Freiss N. et al., Proc. Natl. Acad. Sci., 1997, 94, 5249-5254; Semin Cancer Biol 1999, vol 9, p. 3). As a result, HLA-G proteins have been proposed for treating graft rejection in allogeneic or xenogenic organ/tissue transplantation. HLA-G proteins have also been proposed for the treatment of cancers (EP1 054 688), inflammatory disorders (EP1 189 627) and, more generally, immune related diseases. It has also been proposed to fuse HLA-G proteins to specific ligands in order to target HLA-G to particular cells or tissues (WO2007091078). It should be noted, however, that no results or experimental data have been provided to show that such targeting fusions are active.

At present, it is not clear what conformation is the most active for pharmaceutical purpose, how soluble forms of HLA-G can be used, nor which domains of HLA-G are required for most effective therapy.

### SUMMARY OF THE INVENTION

The present invention relates to novel proteins or polypeptides, pharmaceutical compositions comprising the same, and the uses thereof. More specifically, the present invention relates to a novel polypeptide comprising the sequence of an HLA-G5 antigen fused to the sequence of a B2 microglobulin. As shown in the experimental section, this polypeptide is biologically active in vivo, can form dimers, and can produce a strong immune response in a model of graft rejection. This polypeptide thus represents a drug candidate for treating such disorders, as well as other immune-related diseases.

An object of the present invention thus resides in a fusion polypeptide comprising the sequence of a β2 microglobulin fused to the sequence of an HLA-G5 antigen. In a preferred embodiment, the HLA-G5 antigen is a human HLA-G5 antigen. Furthermore, in a preferred embodiment, the sequence of the β2 microglobulin is fused to the sequence of the HLA-G5 antigen through a spacer group and/or is located on the N-terminal part of the polypeptide.

A preferred object of this invention is a polypeptide comprising the following structure:

B2M—SPACER—HLA-G5

wherein B2M is the sequence of a β2 microglobulin; SPACER is the sequence of a spacer group comprising preferably from 5 to 20 amino acid residues; and HLA-G5 is the sequence of an HLA-G5 antigen.

A further object of this invention resides in a nucleic acid molecule encoding a polypeptide of this invention.

The invention also relates to a vector comprising a nucleic acid molecule as defined above.

Another object of this invention is a recombinant host cell comprising a nucleic acid molecule or a vector as defined above.

A further object of this invention is a method of producing a polypeptide as defined above, comprising culturing a recombinant host cell of the invention under conditions allowing expression of the nucleic acid molecule, and recovering the polypeptide produced.

The invention further relates to a dimer (e.g., a homodimer or a heterodimer) of a polypeptide of the invention.

The invention also relates to an antibody that specifically binds a fusion polypeptide of this invention or a dimer thereof.

The invention also relates to a pharmaceutical composition comprising a polypeptide as defined above, either as a monomer or as a multimer.

The invention also relates to a pharmaceutical composition comprising a nucleic acid encoding a polypeptide as defined above, or a recombinant cell expressing such a polypeptide.

The invention further relates to such polypeptides or pharmaceutical compositions for treating organ or tissue rejection, inflammatory diseases or auto-immune diseases.

A further objet of this invention also relates to a method of treating organ/tissue rejection, the method comprising administering to a subject in need thereof an effective amount of a polypeptide or composition of this invention. More specifically, the method comprises administering the polypeptide or composition to the subject, prior to, during and/or after tissue/organ transplant.

A further object of this invention is a method of promoting tolerance to graft in a subject, the method comprising administering to a subject in need thereof an effective amount of a polypeptide or composition as defined above.

The invention may be used in any mammalian subject, preferably in human subjects. As will be further disclosed below, the polypeptides of this invention are able to substantially inhibit tissue rejection in vivo following allogeneic or xenogenic transplantation.

### LEGEND TO THE FIGURES

FIG. 1: HLA-G5-B2M can form dimers.

FIG. 2: HLA-G5-B2M promotes graft survival in vivo.

### DETAILED DESCRIPTION OF THE INVENTION

The present invention relates to fusion polypeptides comprising an HLA-G5 antigen fused to a B2M. The fusion polypeptides of this invention are biologically active and have been shown to effectively inhibit graft rejection in vivo. More specifically, the inventors have found that, by fusing an HLA-G5 antigen to a B2M, biologically active proteins are obtained which exhibit high immune effect. The results presented in this application show that such a polypeptide can promote graft tolerance in vivo very efficiently and therefore represents a novel medicament for treating immune-related disorders, particularly for reducing unwanted or deleterious immune responses in a subject.

A first object of the present invention thus resides in a polypeptide comprising the amino acid sequence of a B2M fused to the amino acid sequence of an HLA-G5 antigen.

Within the context of the present invention, the terms “polypeptide” and “protein” designate, interchangeably, a molecule comprising a polymer of amino acid residues, which may be linked together through amine linkage, or through modified, peptidomimetic linkages. The amino acid residues in said proteins or polypeptides may be either natural amino acid residues, or non-natural or modified amino acid residues. They may be in L and/or D conformation. Also, the polypeptide or protein may be terminally protected and/or modified, e.g., through chemical or physical alteration of lateral functions, for instance.

Within a polypeptide of this invention, the various polypeptide domains are covalently linked together so that they can, most preferably, be produced as a single molecule through recombinant techniques, i.e., they are fused by an amine linkage.

In the proteins of this invention, the B2M sequence is located N-ter of the HLA-G5 sequence and they are linked together through a spacer group, according to the following structure:

B2M—SPACER—HLA-G5

wherein B2M is the sequence of a β2 microglobulin; SPACER is the sequence of a spacer group; and HLA-G5 is the sequence of an HLA-G5 antigen.

B2 microglobulin (“B2M”) is a serum protein of 11,8 KDa which is found in association with the major histocompatibility complex (MHC) class I heavy chain on the surface of nearly all nucleated cells. Beta-2-microglobulin is essential to the cell surface expression of HLA molecules.

A specific example of a sequence of B2M is disclosed in SEQ ID NO: 2 (see amino acid residues 21-119). It should be understood that alternative B2M sequences may be obtained from genebank or from other publications (see, for instance, Genebank number NC 000015, GeneID:567, First publication of the sequence: Gussow et al., 1987 [PubMed 3312414]). Furthermore, natural variants of B2M exist, e.g., as a result of polymorphism, which are included in the present application. Also, variants of the above sequences which lack certain (e.g., between 1 and 10, preferably between 1-5, most preferably 1, 2, 3, 4 or 5) amino acid residues, and/or contain certain (e.g., between 1 and 10, preferably between 1-5, most preferably 1, 2, 3, 4 or 5) amino acid substitutions or insertions are also included in the present invention.

The spacer group designates any group (e.g., a peptide) which allows a proper refolding of the polypeptide. The spacer can have a variable length and should preferably be biologically inert. Typically the spacer is a peptide of from 8 to 20 amino acid residues in length, more preferably from 8 to 15, even more preferably from 12 to 15. In a specific embodiment, the spacer group has the sequence (G4S)n, wherein n is 2 or 3.

The HLA-G5 domain contained in the polypeptide comprises the amino acid sequence of an HLA-G5 antigen, preferably of a human HLA-G5 antigen, or variants thereof. As indicated before, the HLA-G5 protein isoform is a soluble protein containing the β1, β2 and β3 domains, as well as an extra C-terminal peptide sequence of 21 amino acid residues encoded by intron 4 (as a result of a modification of the reading frame). HLA-G5 does not contain a transmembrane domain or a cytoplasmic domain.

Within the context of this invention the term “HLA-G5 antigen” therefore designates a polypeptide containing the sequence of β1, β2 and β3 domains of a HLA-G antigen and lacking a transmembrane and a cytoplasmic domain. In a preferred embodiment, the HLA-G5 further comprises an additional the C-terminal peptide sequence of 21 amino acid residues encoded by intron 4 of HLA-G.

A specific example of an HLA-G5 sequence is a sequence consisting of the β1, β2 and β3 domains, as well as the C-terminal peptide sequence of 21 amino acid residues encoded by intron 4.

An example of the amino acid sequence of a human HLA-G5 antigen is provided in SEQ ID NO: 2 (see amino acid residues 135-end). Other HLA-G5 sequences are available on line (see e.g., Fujii, T et al, Journal of Immunology, 1994. PMID: 7989753) or in e.g., U.S. Pat. No. 5,856,442 and U.S. Pat. No. 6,291,659.

It should be understood that natural variants of HLA-G5 exist, e.g., as a result of polymorphism, which are included in the present application. Also, variants of the above sequences which lack certain (e.g., between 1 and 10, preferably between 1 and 5, most preferably 1, 2, 3 or 4) amino acid residues, and/or contain certain (e.g., between 1 and 10, preferably between 1 and 5, most preferably 1, 2, 3 or 4) amino acid substitutions or insertions are also included in the present invention.

A specific example of a fusion polypeptide of the invention is HLA-G5-B2M of SEQ ID NO: 2. In HLA-G5-B2M, the amino acid sequence of human B2M is located on the N-ter side of the polypeptide and is linked to the sequence of a human HLA-G5 antigen through a (G4S)3 spacer group. HLA-G5-B2M further comprises a leader peptide sequence of B2M (amino acid residues 1-20), allowing secretion of the polypeptide.

A further specific polypeptide of this invention is a polypeptide comprising amino acid residues 21-END of SEQ ID NO: 2 (i.e., lacking a leader peptide sequence).

As mentioned in the examples, such a B2M-HLA-G5 polypeptide is able to promote graft tolerance in vivo.

In this respect, the invention demonstrates, for the first time, that HLA-G antigens can be fused to a B2M sequence through specific molecular arrangement to produce fully active biological molecules. The invention thus also resides in a polypeptide having the following structure:

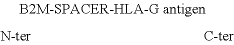

wherein B2M is the sequence of a β2 microglobulin as defined above; SPACER is a spacer group as defined above; and HLA-G is the sequence of an HLA-G antigen. The sequence of the HLA-G antigen typically comprises the sequence of at least the α1 domain of an HLA-G antigen, preferably of a human HLA-G antigen.

A further object of this invention is a dimer of a polypeptide as defined above. The dimer may be a homodimer, e.g., between two identical polypeptides, or a heterodimer, e.g., a dimer comprising at least one polypeptide of this invention.

The polypeptides of this invention can be obtained using techniques known per se in the art, such as artificial synthesis, recombinant techniques, and/or combinations thereof In a typical embodiment, as illustrated in the examples, the polypeptide is produced by recombinant techniques, starting from a chimeric coding polynucleotide.

In this respect, a further object of this invention is a nucleic acid molecule encoding a polypeptide as defined above. The nucleic acid may be e.g., RNA or DNA, single- or double-stranded. It may be produced by techniques known per se in the art, such as genetic engineering, chemical or enzymatic synthesis, etc. In a particular embodiment, the nucleic acid further comprises a sequence encoding a leader peptide for secretion, operably linked to the sequence encoding the polypeptide. As a result, expression of such a nucleic acid leads to the secretion of the polypeptide by the selected host cell. The leader peptide may by of various origins, such as from human or mammalian genes, e.g., B2M, interleukin, HLA-G, etc. A specific example of a nucleic acid of this invention comprises SEQ ID NO: 1 (or nucleotide residues 61-END thereof, i.e., without the leader sequence).

A further object of this invention also resides in a vector comprising a nucleic acid as defined above. The vector may be a cloning and/or expression vector, such as a plasmid, cosmid, phage, a viral vector, an artificial chromosome, etc. Specific examples of such vectors include pFUSE plasmids, pUC plasmids, pcDNA plasmids, pBR plasmids, retroviral vectors, adenoviral vectors, baculoviral vectors, lambda phage vectors, etc. The vector may comprise regulatory sequences, such as a promoter, a terminator, an origin of replication, etc. The vector may be used to produce polypeptides of this invention in vitro, by recombinant techniques, or directly in vivo, in gene therapy approaches.

A further object of this invention is a recombinant host cell comprising a nucleic acid or a vector as defined above. The host cell may be prokaryotic or eukaryotic. Examples of prokaryotic hosts include any bacteria, such as E. coli. Examples of eukaryotic cells include yeasts, fungi, mammalian cells, plant cells or insect cells. Recombinant cells of this invention may be prepared by transformation techniques known per se in the art, such as transfection, lipofection, electroporation, protoplast transformation, etc. These cells may be maintained and cultured in any suitable culture media.

Recombinant cells of this invention can be used e.g., to produce polypeptides of this invention in vitro or ex vivo, or as cell therapy products, to produce the polypeptides in vivo.

In this respect, an object of this invention also resides in a method of producing a polypeptide as disclosed above, the method comprising culturing a recombinant host cell of the invention under conditions allowing expression of the nucleic acid molecule, and recovering the polypeptide produced. The polypeptide may be recovered and/or purified using techniques known per se in the art, such as centrifugation, filtration, chromatographic techniques, etc.

Upon production, the polypeptides of this invention may be modified to improve their properties, for instance to improve their pharmaco-kinetic properties. In this respect, they may be modified to increase their stability or resistance to protease, such as by adding terminal protecting groups (e.g., amide, ester). They may also be coated on a carrier support to increase the polypeptide density.

A further object of this invention is a pharmaceutical composition comprising a polypeptide as defined above and, preferably, a pharmaceutically acceptable excipient or carrier.

A further object of this invention is a pharmaceutical composition comprising a nucleic acid as defined above and, preferably, a pharmaceutically acceptable excipient or carrier.

A further object of this invention is a pharmaceutical composition comprising a recombinant cell as defined above and, preferably, a pharmaceutically acceptable excipient or carrier.

Suitable excipients or carriers include any pharmaceutically acceptable vehicle such as buffering agents, stabilizing agents, diluents, salts, preservatives, emulsifying agents, sweeteners, etc. The excipient typically comprises an isotonic aqueous or non aqueous solution, which may be prepared according to known techniques. Suitable solutions include buffered solutes, such as phosphate buffered solution, chloride solutions, Ringer's solution, and the like. The pharmaceutical preparation is typically in the form of an injectable composition, preferably a liquid injectable composition, although other forms may be contemplated as well, such as tablets, gelules, capsules, syrups, etc. The compositions of this invention may be administered by a number of different routes, such as by systemic, parenteral, oral, rectal, nasal or vaginal route. They are preferably administered by injection, such as intravenous, intraarterial, intramuscular, intraperitoneal, or subcutaneous injection. Transdermal administration is also contemplated. The specific dosage can be adjusted by the skilled artisan, depending on the pathological condition, the subject, the duration of treatment, the presence of other active ingredients, etc. Typically, the compositions comprise unit doses of between 10 ng and 1 mg of fusion polypeptide, more preferably between 10 ng and 100 mg, even more preferably between 10 μg and 100 mg.

The compositions of the present invention are preferably administered in effective amounts, i.e., in amounts which are, over time, sufficient to at least reduce or prevent disease progression. In this regard, the compositions of this invention are preferably used in amounts which allow the reduction of a deleterious or unwanted immune response in a subject.

As mentioned above, the polypeptides of this invention have strong immune-regulatory activity and may be used to treat a variety of disease conditions associated with abnormal or unwanted immune response. More specifically, the polypeptides of this invention are suitable for treating immune-related disorders such as, particularly, organ or tissue rejection, inflammatory diseases or auto-immune diseases.

As disclosed in the experimental section, the polypeptides of this invention can substantially inhibit allogeneic or xenogenic graft rejection in vivo.

An object of the present invention thus resides in a polypeptide or composition as disclosed above for treating graft rejection.

A further object of this invention resides in a method of treating graft rejection in a subject, the method comprising administering to a subject in need thereof an effective amount of a composition as disclosed above.

The term treating designates for instance the promotion of the graft tolerance within the receiving subject. The treatment can be performed prior to, during and/or after the graft, and may be used as an alternative therapy to existing immunosuppressive agents or, as a combined therapy with actual immunosuppressive agents. The invention is applicable to allogenic, semi-allogenic or even xenogenic transplantation, and may be used for any type of transplanted organs or tissues including, without limitation, solid tissues, liquid tissues or cells, including heart, skin, kidney, liver, lung, liver-kidney, etc.

A further object of this invention is an improved method for transplanting an organ or tissue in a subject, the improvement comprising administering to the subject, prior to, during and/or after transplantation, an effective amount of a composition as disclosed above.

A further object of this invention is a method for promoting graft tolerance in a subject, the method comprising administering to the subject, prior to, during and/or after transplantation, an effective amount of a composition as disclosed above.

A further object of this invention is a method for reducing graft rejection in a subject, the method comprising administering to the subject, prior to, during and/or after transplantation, an effective amount of a composition as disclosed above.

A further object of the present invention resides in a polypeptide or composition as disclosed above for treating an auto-immune disease. The invention also resides in a method of treating an autoimmune disease in a subject, the method comprising administering to a subject in need thereof an effective amount of a composition as disclosed above. The autoimmune disease may be Rheumatoid arthritis, Crohn's disease or multiple sclerosis. In such disease conditions, the invention allows to reduce the deleterious immune response which is responsible for the pathology.

Another object of the present invention resides in a polypeptide or composition as disclosed above for treating an inflammatory disease.

A further object of this invention resides in a method of treating an inflammatory disease in a subject, the method comprising administering to a subject in need thereof an effective amount of a composition as disclosed above.

It should be understood that the amount of the composition actually administered shall be determined and adapted by a physician, in the light of the relevant circumstances including the condition or conditions to be treated, the exact composition administered, the age, weight, and response of the individual patient, the severity of the patient's symptoms, and the chosen route of administration. Therefore, the above dosage ranges are intended to provide general guidance and support for the teachings herein, but are not intended to limit the scope of the invention.

Further aspects and advantages of this invention will be disclosed in the following examples, which should be considered as illustrative and not limiting the scope of this application.

### EXAMPLES

Materials and Methods

Amplification by PCR

PCR were performed on a GeneAmp PCR System 9600 (Perkin Elmer) in a final volume of 50 μl containing 20 ng of DNA, 200 nM of each primer, 200 μM of dNTP (Invitrogen), 2.5 μl of PCR Buffer 10× (Perkin Elmer), 2,5 Unit of Tag polymerase (Perkin Elmer) and 27 μl of water.

Program used was the following:

DNA denaturation during 5 minutes at 94° C. followed by 30 cycles of:
30 seconds at 94° C.
30 seconds at 58° C.
1 minute at 72° C.
At the end of the last cycle a 5 minutes step at 72° C. was performed

Vectors

pFUSE-hFc1 and pFuse-mFc2 vector were both purchased from the company InvivoGen.

Enzymatic Digestion

Enzymes restriction digestions were performed as recommended by the manufacturer (invitrogen). Typically, digestions were performed for 1 hour at 37° C. with 1 μg of DNA and 5 unit of restriction enzymes in the adequate buffer.

Ligations

Ligation of PCR fragments into expression vector were performed with the T4 DNA ligase from Promega as recommended by the manufacturer. For HLA-G5-beta-2 microglobulin construction ligation of PCR fragment into pcDNA 3.1 D/V.5-His-Topo (Invitrogen) was performed directly with the 3.1 Directional TOPO® Expression Kit (Invitrogen)

Plasmid Purification

Plasmid purification were performed with the GenElute™ Plasmid Midiprep (Sigma) as recommended by the manufacturer.

Protein Production

For production of the fusion protein, HEK293T or HELA cells were transfected by the diverse constructs with the lipofectamine method (invitrogen) and kept at 37° C., 5% CO2 in DMEM (Dulbco's Modified EagleMedium) supplemented with 10% foetal calf serum and 0.3M glutamine. After 48 hours supernatant were harvested, filtrated through 0.2 μM filter and then used for experiments or to prepare stocks.

### Example 1

### Cloning and Synthesis of HLA-G5-B2M

Beta-2-microglobulin fused to α1, α2 and α3 domain was amplified by PCR using plasmid pFUSE-hFc1-HLAG1-B2M as template (unpublished) with primers

[symbol]

B2Msig TOPO sens

[symbol]

### 5′ CACCATGTCTCGCTCCGTGGCC (SEQ ID NO: 3) and

alpha3-i4-Xho-Stop anti sens [symbol]
5′ ATC TTA ACT CGA GAG GTC TTC AGA GAG GCT CCT GCT TTC CCT AAC AGA CAT GAT GCC TCC ATC TCC CTC CTT ACT CCA TCT CAG CAT GAG 3′ (SEQ ID NO: 4) that contains intron 4 sequence from HLA-G5. The PCR fragment was then ligated into the pcDNA 3.1 D/V.5-His-Topo vector (Invitrogen) using 3.1 Directional TOPO® Expression Kit (Invitrogen).

The resulting cDNA sequence is described in SEQ ID NO: 1. The amino acid sequence of the protein is described in SEQ ID NO: 2.

The protein was produced as disclosed in the materials and methods.

### Example 2

### HLA-G5-B2M Forms Dimers

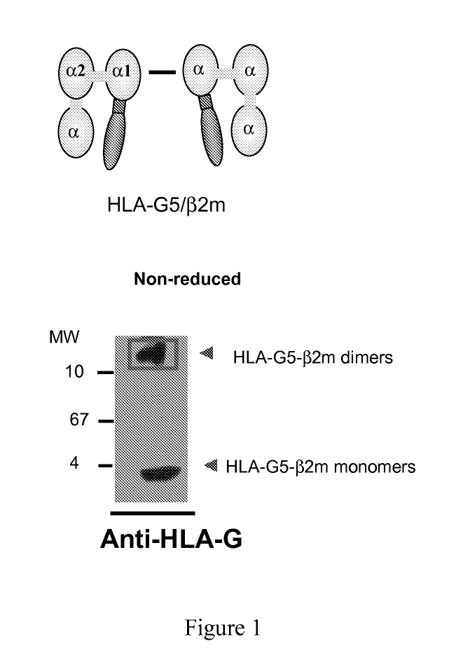

FIG. 1 represents HLA-G5-beta-2-microglobulin dimers (upper band) and monomers (lower band) protein migration by PolyAcrylamide Gel Electrophoresis. HLA-G5-beta-2-microglobulin protein present in supernatant was immunoprecipitated with Protein G sepharose beads (GE Healthcare) previously coated with anti-HLA-G5 antibody (MEMG/09). Immunoprecipitates were washed three times with PBS 1×. Proteins were then eluted by incubation with sample buffer in non-reducing condition, boiling, electrophoresed on polyacrylamide gels and transferred onto Hybond ECL nitrocellulose membranes (Amersham Pharmacia Biosciences). Following incubation with 5% non-fat milk in PBS 1×, the membrane was incubated overnight with anti-HLA-G (4H84) antibody and revealed using HorseRadish peroxydase-conjugated goat anti-mouse secondary antibody. Membranes were revealed with ECL detection system (Amersham Pharmacia Biosciences).

The results presented demonstrate the ability of fusion proteins of this invention to form dimers.

### Example 3

### HLA-G5-B2M Promotes Survival of Allogeneic Skin Transplant in vivo

Material

Sulfate latex beads 4% w/v 5 μm (Invitrogen)
AffiniPure Coat Anti-mouse IgG Fc Fragment 1.8 mg/ml (Jackson ImmunoResearch)
AffiniPure Coat Anti-human IgG Fc Fragment 1.3 mg/ml (Jackson ImmunoResearch)

### HeLa Negative Control

HLAG5-b2m 1.5 μg/ml

Method

For every HLA-G/Fc fusion protein, 10⁸ Sulfate latex beads were coated with 20 μg/ml AffiniPure Coat Anti-mouse (or anti-human) IgG Fc Fragment 2 hr at 37° C. followed by 2 hr incubation with BSA (2 mg/ml). After washing, the beads were incubated with 0.5 μg/ml of HLA-G/Fc fusion proteins at 4° C. for 16 hr. Subsequently, the beads were washed 2 times by 1× PBS. 5 ml of HLA-G/Fc fusion proteins (1 μg/ml) was used for 5× 10⁶ sulfate latex beads. As a negative control, sulfate latex beads were prepared in an identical manner except that 1×PBS or HeLa Negative Control was used rather than HLA-G/Fc fusion proteins. Sulfate latex beads (5×10⁶) were injected intraperitoneal (i.p.) on the day before skin grafting.

Specific pathogen-free C57BK/16 (H-2b) mice and ILT4-transgenic mice (H-2b) (8-10 weeks of age) were used as skin graft recipients throughout the study. Recipient mice received HLA-G-coupled microspheres, Donor skin was from MHC class II-disparate B6.CH-2bm12 (bm12, H-2b) mice. Allogeneic skin grafts have been performed by standard methods. Briefly, skin (1.0 cm²) from the tail of donor mice (12-14 weeks old) was grafted onto the flank of recipient, anesthetized mice. The graft was covered with gauze and plaster, which was removed on day 10. Grafts were scored daily until rejection (defined as 80% of grafted tissue becoming necrotic and reduced in size). All skin grafting survival data were tested by Kaplan Meier Survival Analysis.

### Results

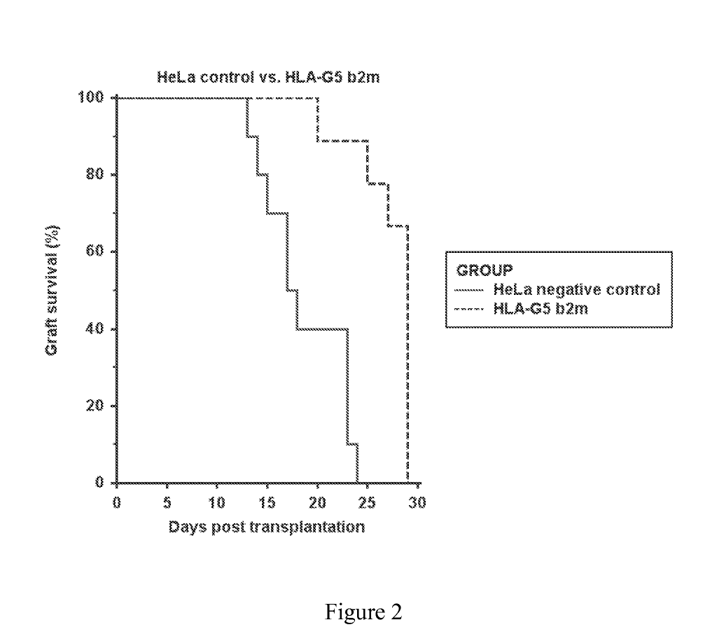

The results are depicted on FIG. 2. They show that HLA-G5-B2M was able to substantially improve graft tolerance in vivo. It should be noted that each day of graft survival in the model corresponds to approximately at least one month of graft survival in human subjects.

SEQUENCE LISTING

SEQ ID NO: 1: cDNA
seq signal B2m/Beta2m/Linker/Gα1/Gα2/Gα3/intron4
ATG TCT CGC TCC GTG GCC TTA GCT GTG CTC GCG CTA CTC TCT CTT
TCT GGC CTG GAG GCT ATC CAG CGT ACT CCA AAG ATT CAG GTT TAC
TCA CGT CAT CCA GCA GAG AAT GGA AAG TCA AAT TTC CTG AAT TGC
TAT GTG TCT GGG TTT CAT CCA TCC GAC ATT GAA GTT GAC TTA CTG
AAG AAT GGA GAG AGA ATT GAA AAA GTG GAG CAT TCA GAC TTG TCT
TTC AGC AAG GAC TGG TCT TTC TAT CTC TTG TAC TAC ACT GAA TTC
ACC CCC ACT GAA AAA GAT GAG TAT GCC TGC CGT GTG AAC CAT GTG
ACC TTG TCA CAG CCC AAG ATA GTT AAG TGG GAT CGA GAC ATG GGA
GGT GGC GGA TCC GGA GGT GGC GGA TCC GGA GGT GGC GGA TCC GGC
TCC CAC TCC ATG AGG TAT TTC AGC GCC GCC GTG TCC CGG CCC GGC
CGC GGG GAG CCC CGC TTC ATC GCC ATG GGC TAC GTG GAC GAC ACG
CAG TTC GTG CGG TTC GAC AGC GAC TCG GCG TGT CCG AGG ATG GAG
CCG CGG GCG CCG TGG GTG GAG GAG GAG GGG CCG GAG TAT TGG GAA
GAG GAG ACA CGG AAC ACC AAG GCC CAC GCA CAG ACT GAC AGA ATG
AAC CTG CAG ACC CTG CGC GGC TAC TAC AAC CAG AGC GAG GCC AGT
TCT CAC ACC CTC CAG TGG ATG ATT GGC TGC GAC CTG GGG TCC GAC
GGA CGC CTC CTC CGC GGG TAT GAA CAG TAT GCC TAC GAT GGC AAG
GAT TAC CTC GCC CTG AAC GAG GAC CTG CGC TCC TGG ACC GCA GCG
GAC ACT GCG GCT CAG ATC TCC AAG CGC AAG TGT GAG GCG GCC AAT
GTG GCT GAA CAA AGG AGA GCC TAC CTG GAG GGC ACG TGC GTG GAG
TGG CTC CAC AGA TAC CTG GAG AAC GGG AAG GAG ATG CTG CAG CGC
GCG GAC CCC CCC AAG ACA CAC GTG ACC CAC CAC CCT GTC TTT GAC
TAT GAG GCC ACC CTG AGG TGC TGG GCC CTG GGC TTC TAC CCT GCG
GAG ATC ATA CTG ACC TGG CAG CGG GAT GGG GAG GAC CAG ACC CAG
GAC GTG GAG CTC GTG GAG ACC AGG CCT GCA GGG GAT GGA ACC TTC
CAG AAG TGG GCA GCT GTG GTG GTG CCT TCT GGA GAG GAG CAG AGA
TAC ACG TGC CAT GTG CAG CAT GAG GGG CTG CCG GAG CCC CTC ATG
CTG AGA TGG AGT AAG GAG GGA GAT GGA GGC ATC ATG TCT GTT AGG
GAA AGC AGG AGC CTC TCT GAA GAC CTC
SEQ ID NO: 2: Amino acid sequence of HLA-G5/β2m.
seq signal B2m: residues 1-20
Beta2m: residues 21-119
Linker: residues 120-134
Gα1/Gα2/Gα3/intron4: residues 135-END
M S R S V A L A V L A L L S L S G L E A I Q R I P K I Q V Y
S R H P A E N G K S N F L N C Y V S G F H P S D I E V D L L
K N G E R I E K V E H S D L S F S K D W S F Y L L Y Y T E F
T P T E K D E Y A C R V N H V T L S Q P K I V K W D R D M G
G G G S G G G G S G G G G S G S H S M R Y F S A A V S R P G
R G E P R F I A M G Y V D D T Q F V R F D S D S A C P R M E
P R A P W V E E E G P E Y W E E E T R N T K A H A Q T D R M
N L Q T L R G Y Y N Q S E A S S H T L Q W M I G C D L G S D
G R L L R G Y E Q Y A Y D G K D Y L A L N E D L R S W T A A
D T A A Q I S K R K C E A A N V A E Q R R A Y L E G T C V E
W L H R Y L E N G K E M L Q R A D P P K T H V T H H P V F D
Y E A T L R C W A L G F Y P A E I I L T W Q R D G E D Q T Q
D V E L V E T R P A G D G T F Q K W A A V V V P S G E E Q R
Y T C H V Q H E G L P E P L M L R W K A V A S K E G D G G I
M S V R E S R S L S E D L

1-15. (canceled)

16. A polypeptide comprising, in the N-ter->C-ter orientation, the amino acid sequence of a β2 microglobulin, a spacer group and the amino acid sequence of an HLA-G5 antigen.

17. The polypeptide according to claim 16, wherein the β2 microglobulin is a human b2 microglobulin.

18. The polypeptide according to claim 16, wherein the β2 microglobulin comprises amino acid residues 21-119 of SEQ ID NO: 2.

19. The polypeptide according to claim 16, wherein the HLA-G5 antigen is a human HLA-G5 antigen.

20. The polypeptide according to claim 16, wherein the HLA-G5 antigen is selected from the group consisting of:
a) the amino acid sequence of the α1, α2 and α3 domains of an HLA-G antigen;
b) the amino acid sequence of the α1, α2 and α3 domains and of the 21 amino acid residues encoded by intron 4 of an HLA-G antigen;
c) the amino acid residues 135-END of SEQ ID NO: 2; and
d) an amino acid sequence as defined in any one of a) to c) having from 1 to 5 amino acid deletion, substitution or insertion.

21. The polypeptide according to claim 16, wherein the spacer group is a peptide of 8 to 20 amino acid residues or a (G4S)n sequence, wherein n is 2 or 3.

22. The polypeptide according to claim 16, said polypeptide comprising SEQ ID NO: 2 or amino acid residues 21-END thereof

23. A nucleic acid molecule encoding a polypeptide according to claim 16.

24. A vector comprising a nucleic acid molecule according to claim 23.

25. A recombinant host cell comprising a nucleic acid molecule according to claim 23.

26. A method of producing a polypeptide comprising culturing a recombinant host cell of claim 25 under conditions allowing expression of the nucleic acid molecule, and recovering the polypeptide.

27. A dimer of a polypeptide according to claim 16.

28. A pharmaceutical composition comprising a polypeptide according to claim 16.

29. A method of treating organ or tissue rejection comprising the administration of a composition according to claim 28 to a subject.

30. A method of treating inflammatory disease or an autoimmune disease comprising the administration of a composition according to claim 28 to a subject.
